(19) United States
(12) Patent Application Publication
(10) Pub. No.: US 2023/0337868 A1
(43) Pub. Date: Oct. 26, 2023

(54) HAND SANITIZER

(71) Applicant: QUANTUM LEAP LLC, Miami, FL (US)

(72) Inventors: Frank R. Ruiz, Casa Grande, AZ (US); Henry A. Ruiz, Long Beach, CA (US); Robert J. Fulbright, Gilbert, AZ (US); Daniel G. Reed, Mesa, AZ (US); Roberto M. Suárez, Miami, FL (US); William R. Trueba, Miami, FL (US)

(73) Assignee: QUANTUM LEAP LLC, Miami, FL (US)

(21) Appl. No.: 17/922,810

(22) PCT Filed: Mar. 11, 2021
(86) PCT No.: PCT/US21/22010

§ 371 Date: Nov. 2, 2022

Related U.S. Application Data
(60) Provisional application No. 62/988,255, filed on Mar. 11, 2020.

(51) Int. Cl. A47K 5/12; A47K 7/04; B05B 17/06

(52) U.S. Cl. CPC A47K 5/1217; A47K 5/1202; A47K 7/04; B05B 17/0661

(57) ABSTRACT

The present invention provides an apparatus for the delivery of a mist-borne solution substantially contained within a predefined volume. The apparatus thus disclosed is comprised of an active delivery area (herein referred to as a “predefined volume”), at least one detection sensor, a chamber or other suitable area within which to contain an amount of a solution in a liquid state, at least one piezoelectric transducer, and a microcontroller.

### CROSS-REFERENCE TO RELATED APPLICATIONS

This application is a non-provisional of, and claims priority to, U.S. Provisional Patent Application No. 62/988,255, “Hand Sanitizer,” filed on Mar. 11, 2020, and incorporated by reference as if fully set forth herein.

### FIELD OF THE INVENTION

The present invention generally relates to devices and methods directed to the delivery of mist-borne solutions, and more particularly relates to an apparatus and related method for delivering hand sanitizer onto hands using a substantially captured mist-borne sanitizer solution.

### BACKGROUND OF THE INVENTION

It is common knowledge that germs can be spread from person to person by touching a contaminated surface. The age-old answer to this problem has been to wash your hands with soap and water. This low-tech solution works just fine at home, but when you are in a public place, washing your hands with soap and water means that you have to dry your hands afterwards. Commercial hand dryers dry your hands without need of a towel, but they have drawbacks. Often, the moisture from wet hands collects in cracks at the bottom of the dryer and can form mold which can then be blown back out into the ambient air. Sometimes commercial hand dryers can also spread pathogens because they circulate air near the device, including air that comes into contact with pathogens near the device or on surfaces near the device. This is often true in bathrooms or restrooms.

Certain solutions are able to kill germs and these solutions can be easily and economically dispensed as hand sanitizers, requiring no hand drying afterwards. For this reason, hand sanitizers have become ubiquitous. We see them in super markets, hospital emergency rooms, schools, gyms, public transportation, and just about any high traffic places. Many hand sanitizer gels are sold in “pocket packs” for personal use. These usually contain a high percentage of alcohol. The alcohol-based gels take approximately 10-15 seconds to dry on the hands. Once dried, the alcohol-based gels, foam, and wipes lose their efficacy and have no effect whatsoever against bacteria.

Some hand sanitizers are dispensed from a container affixed to a wall. Depressing a tab on the container causes a glob of sanitizing gel to be released. Other hand sanitizers are in the form of hand sanitizing wipes dispensed out of a pop-up dispenser. These sanitizing methods are easy to use and effective; however, they all suffer from a serious drawback. Namely, the problem with these known methods is that activation requires touch, which introduces a locus of contamination—the actual sanitizer dispenser. The alcohol based sanitizing gel can also be messy, causing droplets to spill. Touch-free soap dispensers address the touch contamination problem by automatically dispensing soap upon sensor activation. A user places his/her hand underneath the dispenser and gel soap is dispensed. The problem with this method is that the dispensers must be re-filled or replaced often. Moreover, the dispensers must be used in conjunction with a supply of water and preferably, a mechanism to dry the hands afterwards.

There exists, therefore, a need for a hand sanitizing system and method to overcome the above-stated shortcomings of the known art.

### SUMMARY

We disclose an apparatus for the delivery of a mist-borne solution substantially contained within a predefined volume. The apparatus thus disclosed is comprised of an active delivery area (herein referred to as a “predefined volume”), at least one detection sensor, a chamber or other suitable area within which to contain an amount of a solution in a liquid state, at least one piezoelectric transducer, and a microcontroller.

The predefined volume is a substantially closed space, having a length, width, and height, within which the mist-borne solution is introduced and substantially contained. This predefined volume may be achieved by using physical structures (such as a container), or by implementing forced air curtains with a sufficiently strong directed airflow such that the mist-borne solution cannot pass through. The predefined volume is, however, permeable or penetrable such that an object to which the mist-borne solution is desired to coat (or be delivered onto) can be inserted and removed from the predefined volume. The object for which the apparatus is configured to deliver the mist borne solution to will generally dictate the size and shape of the predefined volume. By way of example and not limitation, in embodiments where the object is a pair of human hands, then the predefined volume is configured for the ease of insertion and removal of the two hands without the hands needing to touch any surrounding apparatus structure. As used herein, the term “predefined volume” and “chamber” are used interchangeably when referring to the part of the apparatus within which an external object (such as a user's hands) are momentarily inserted for the delivery of the mist-borne solution.

The detection sensor is configured to detect the presence of an object within the predefined volume. The detection sensor may be an optical (visible light) sensor, an infra-red sensor, a motion sensor, or other such sensor capable of detecting the presence of an object within the predefined volume. Preferably, this sensing is accomplished without any physical contact with the object.

The solution to be delivered to the object inserted into the predefined volume is contained, in its liquid state, in a chamber communicative with the predefined volume. By way of example and not limitation, this solution may be an antiseptic and antibacterial hand sanitizing solution that has been specially formulated to form a mist when subjected to ultrasonic frequencies. As used in this disclosure, the term “solution” may refer to a single “ingredient” liquid (such as water or alcohol), or it may also refer to a multi-ingredient liquid solution (such as a benzalkonium chloride aqueous solution), or it may refer to a suspension or emulsion (such as a suspension of water and essential oils). As used in this disclosure, the terms “mist-borne”, “mist”, “vapor”, “fog” and the like are used interchangeable and indicate the state of the solution upon being agitated by the piezoelectric transducer.

The at least one piezoelectric transducer is in electrical communication with the micro-controller and is configured to be capable of producing ultrasonic frequencies in a range such that the vibration of the piezoelectric transducer breaks apart the solution into a fine mist. This fine mist is similar to a vapor or fog but without the heat required to produce steam. The particles of the mist contain the solution (including any suspended additives) and carry the mist-borne solution into the predefined volume where it is delivered to the object inserted within. It is within the scope of this disclosure that the selection, quantity, and frequency of the chosen piezoelectric transducer is to be paired with the particular solution to be used in the apparatus for delivery. While a typical frequency range may be discussed, below, it is contemplated that other solutions may require other frequencies outside of the discussed range. As used in this disclosure, the terms “mister” and “piezoelectric transducer” are used interchangeably.

The micro-controller, in electrical communication with the detection sensor and the at least one piezoelectric transducer, is programmed to continuously monitor the predefined volume using the detection sensor to determine the presence of an object within the predefined volume. When the object enters the predefined volume, the sensor detects its presence and sends a signal to the micro-controller. The micro-controller then activates the at least one piezoelectric transducer, which begins to vibrate at the pre-configured frequency. These vibrations are communicated to the solution, breaking the solution into fine mist particles. The mist-borne solution drifts into the predefined volume, where it is contained, and surrounds the object, thereby delivering the mist-borne solution to the object.

In a non-limiting, preferred embodiment, the apparatus is a hand sanitizing device with a chamber configured for receiving inserted hands. Once inserted, sensors detect the presence of the hands and the micro-controller turns on the piezoelectric transducers, which, in turn, agitate a specially-formulated hand sanitizing solution into a fine mist. The fine mist propagates into the chamber and surrounds the hands, coating every surface of the hands with the mist-borne sanitizing solution. After a prescribed amount of time, the micro-controller turns off the piezoelectric transducers and the user can remove their now-sanitized hands from the chamber. Additional programming in the micro-controller can exhaust the remaining mist-borne solution so that it does not escape from the chamber, or turn on air-foils or air curtains to similarly contain and direct the mist-borne solution.

A related method of using the disclosed invention is also discussed, below.

### BRIEF DESCRIPTION OF THE DRAWINGS

In the accompanying figures, like reference numerals refer to identical or functionally similar elements throughout the separate views. The accompanying figures, together with the detailed description below are incorporated in and form part of the specification and serve to further illustrate various embodiments and to explain various principles and advantages all in accordance with the present invention, in which:

FIG. 1 is a diagram showing a top view of a hand sanitizer apparatus, according to an embodiment of the present disclosure;

FIG. 2 is a front view of the apparatus shown in FIG. 1;

FIG. 3 is a right-side view of the apparatus shown in FIG. 1;

FIG. 4 is a rear view of the apparatus shown in FIG. 1;

FIG. 5 is a cross-section detail view of the hand sanitizer of FIG. 3;

FIG. 6 is a cross-section detail view of the hand sanitizer of FIG. 4;

FIG. 7 is a cross-section of the hand sanitizer while in operation, according to an embodiment of the present disclosure;

FIG. 8 is an operational flow diagram of the operation of the hand sanitizer, according to an embodiment of the present disclosure;

FIG. 9 is a partial cut-away diagram of a hand sanitizer, according to an embodiment of the present disclosure;

FIG. 10 is a diagram of the top assembly of the hand sanitizer of FIG. 9, according to an embodiment of the present disclosure;

FIG. 11 is a diagram of the base plate for the top assembly of FIG. 10, according to an embodiment of the present disclosure;

FIG. 12 shows the short hose adapter of the top assembly of FIG. 10, according to an embodiment of the present disclosure;

FIG. 13 shows the long-side air foil, according to an embodiment of the present disclosure;

FIG. 14 shows the short-side air foil, according to an embodiment of the present disclosure;

FIG. 15 shows the post bracket, according to an embodiment of the present disclosure;

FIG. 16 shows the large hose adapter, according to an embodiment of the present disclosure;

FIG. 17 is a diagram of the top assembly of the hand sanitizer after the adapters are attached, according to an embodiment of the present disclosure;

FIG. 18 is a diagram of the top assembly of FIG. 17, with post brackets, according to an embodiment of the present disclosure;

FIG. 19 shows air foil placement on the top assembly, according to an embodiment of the present disclosure;

FIG. 20 is a diagram of the mid-assembly of the hand sanitizer, according to an embodiment of the present disclosure;

FIG. 21 shows the mid-assembly plate;

FIG. 22 shows the mid-assembly post;

FIGS. 23A, 23B, and 23C show three views of the single jet, according to an embodiment of the present disclosure;

FIG. 24 shows the bowl base;

FIG. 26 shows the post base;

FIG. 27 shows a fan jet;

FIG. 28 shows a pair of misters, according to an embodiment of the present disclosure;

FIG. 29 shows a top-front isometric view of an apparatus according to an embodiment of the present invention;

FIG. 30 shows a front view of the apparatus shown in FIG. 29;

FIG. 31 shows a right view of the apparatus shown in FIG. 29, where the mirror-image left view is not shown for compactness of disclosure;

FIG. 32 shows a back view of the apparatus shown in FIG. 29;

FIG. 33 shows a top view of the apparatus shown in FIG. 29;

FIG. 34 shows a top view of the apparatus shown in FIG. 29;

FIG. 35 shows a cross-sectional view of the apparatus shown in FIG. 29, including a simplified representation of the air curtain and mist-borne solution path into the predetermined volume;

FIG. 36 shows a 3D rendered top isometric view of the apparatus shown in FIG. 29;

FIG. 37 shows a 3D rendered bottom isometric view of the apparatus shown in FIG. 29;

FIG. 38 shows a 3D rendered bottom isometric view of the apparatus shown in FIG. 29, with the optional wall mount; and

FIG. 39 shows a 3D rendered side view of the apparatus shown in FIG. 29, with the optional wall mount.

### DETAILED DESCRIPTION

Referring now to the figures, we describe two non-limiting exemplary embodiments of the present invention.

### First Embodiment

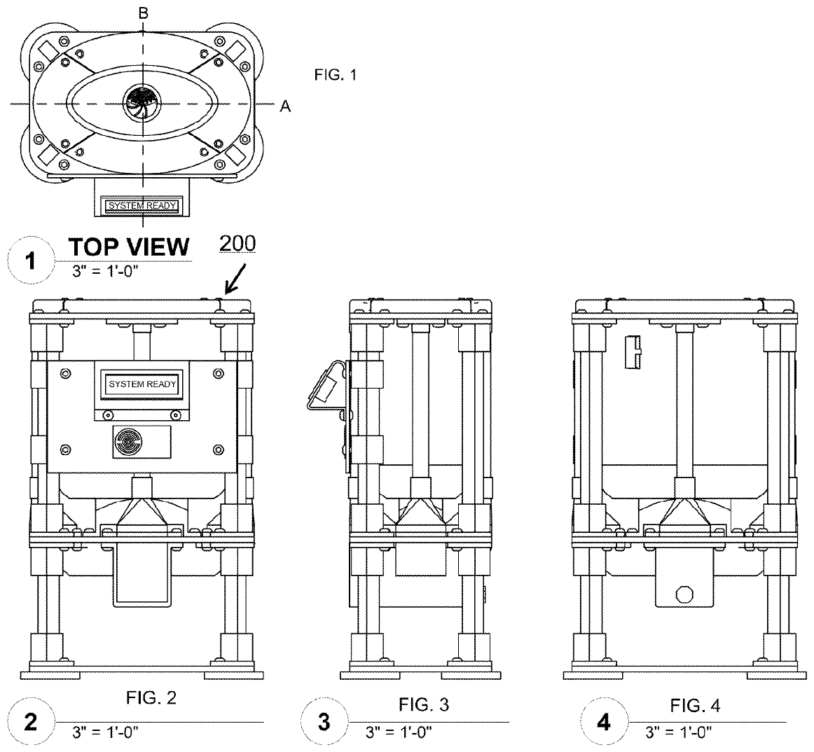

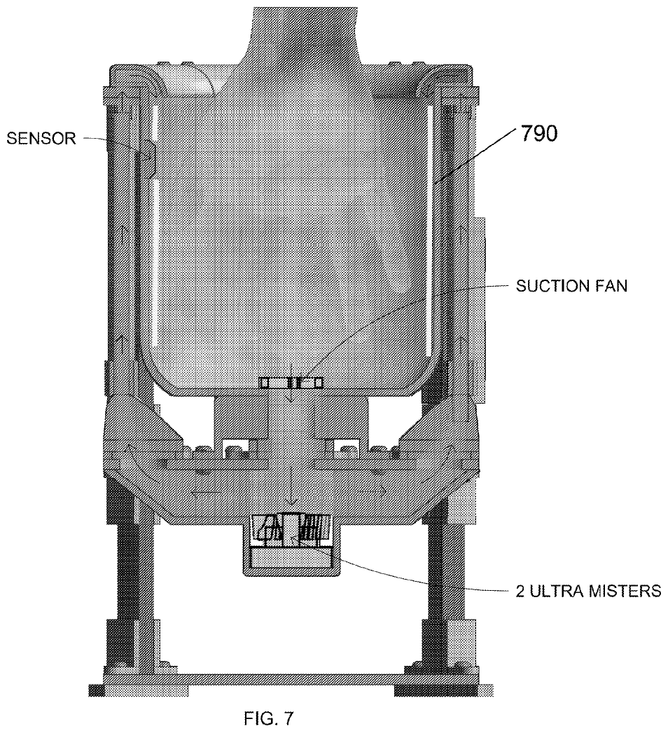

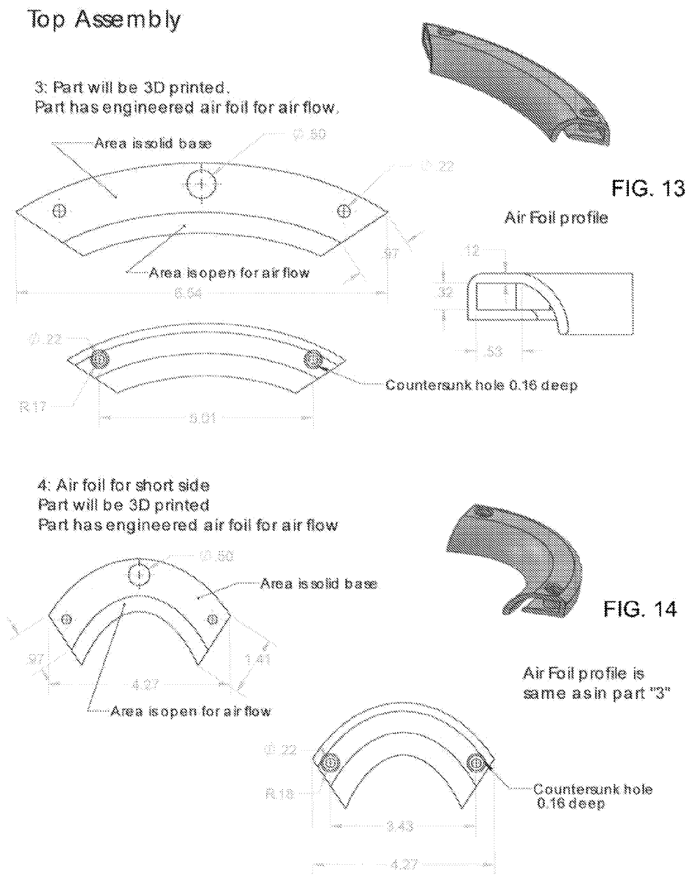

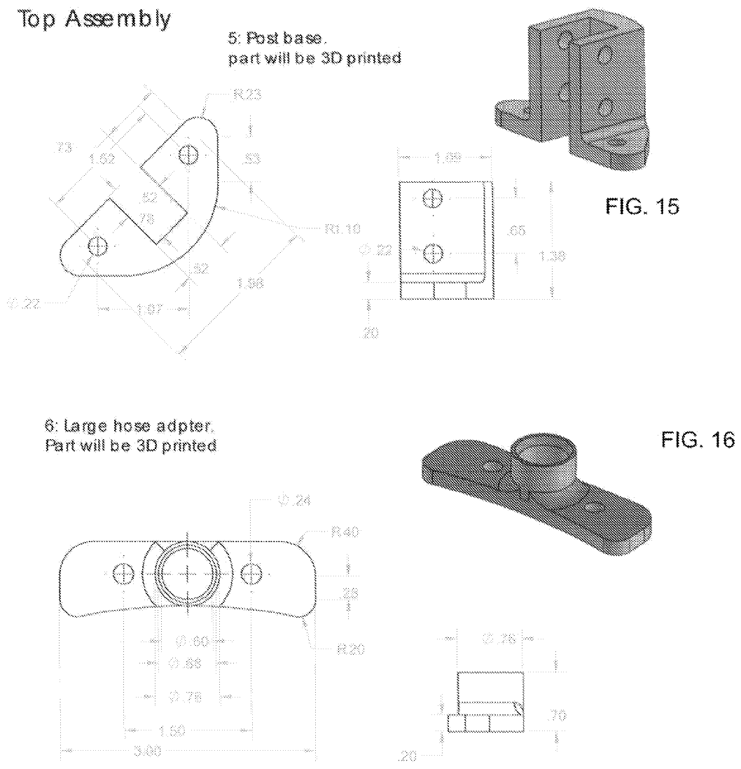

In a first non-limiting embodiment, illustrated in FIGS. 1-28, we describe an embodiment of the apparatus for the delivery of a mist-borne solution substantially contained within a predefined volume where the mist-borne solution is a specially-formulated hand sanitizer and the predefined volume is configured to substantially enclose a hand, once inserted therein (see, for example, FIG. 7).

This unique hand-sanitizing device uses ultrasonic waves generated by the misters to aerosolize specially-formulated hand-sanitizing liquid. The device allows for convenient and economical touch-free hand sanitizing with minimal maintenance required since any remaining vapor is returned back to the device. The device itself operates “hands-free” by dispensing a sanitizing vapor without a user having to touch any part of the device, thereby avoiding touch contamination. The simple act of a user inserting his/her hand into a chamber activates the sanitizing process, coating the user's hand with a sanitizing vapor. The vapor soaks into the skin, delivering bacteria-killing sanitizing benefits much more effectively than gels. After the sanitizing is complete, the user withdraws his/her hand, without the necessity of using towels or paper to dry. The hand-sanitizing liquid continues to kill bacteria on hands for up to 4 hours.

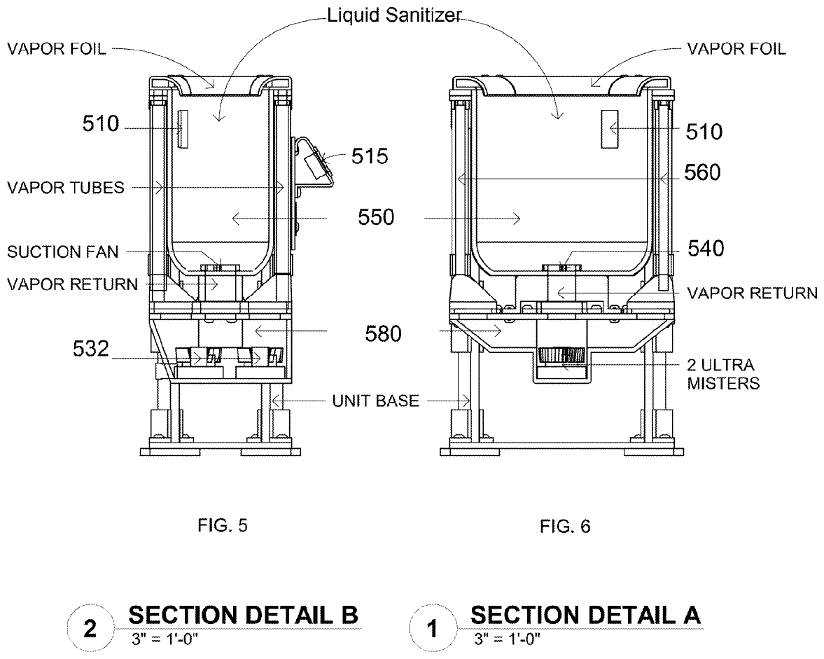

FIGS. 1-4 show several views of the hand sanitizer 200. FIG. 1 shows a top detail view; FIG. 2 shows a front detail view; FIG. 3 shows a side detail view and FIG. 4 shows a back detail view of the hand sanitizer 200. Referring to FIGS. 5 and 6, the hand sanitizer 200 includes, inter alia, a base housing, a base stand, a chamber 550, a reservoir 580, vapor air foils, vapor tubes, a suction fan 540, a vapor return, and ultrasonic misters 532. In further embodiments, the liquid sanitizing solution can be poured directly into the chamber 550.

The hand sanitizer 200 also includes electronic and electrical components configured to produce soundwaves in the ultrasonic frequencies. The base housing is configured to house the chamber 550 and the electronics that are used to create the ultrasonic sound waves. Additional electronics are also contained within the base housing, such as, but not limited to, a power supply/power regulator, a user interface (such as one or more buttons, status lights, and the like), and a fan. By way of example and not limitation, a preferred embodiment of the invention includes one or more piezoelectric disks in electrical communication with a power supply. The piezoelectric disks can be chosen from those known in the art to produce frequencies in the 1.7 MHz to 2.4 MHz range, although, as discussed above, the frequency chosen is to be matched to the frequency required to mist the desired solution. It is contemplated that different solutions may be optimally misted at different frequencies and the piezoelectric transducer should preferably be matched to produce the preferred frequency for the solution.

The device 200 also includes a power supply circuit configured to supply electrical power to the switches, piezoelectric disks, and other electrical and electronic components, such as a microcontroller that can operate 16 mm piezoelectric discs at the required frequency of 1.7-2.4 Mhz for the requisite period of time. The microcontroller may also be specially programmed to drive the piezoelectric transducers at different frequencies as chosen depending on the solution used at the time. In embodiments, the power supply is configured to receive 5V DC power from an external adaptor, but the AC to DC conversion can also be accomplished within the device 200.

In the embodiment of FIGS. 5 and 6, the hand sanitizing liquid is poured directly into the chamber 550. In another embodiment, the hand sanitizing liquid is stored in a container (not shown) that is communicative with the base housing. The container is configured to contain an amount of hand sanitizing solution in sonic communication with the one or more piezoelectric disks. As a precautionary measure to prevent the wrong solution from being used, the sanitizing liquid container is mated with the sanitizer 200 via a proprietary spout.

When the device is activated, it powers the nebulizing disks. The ultrasonic mechanism uses a high frequency to break down (vaporize) the hand-sanitizing solution in the reservoir 580 into a fine mist (2-5 microns in size). Misters 532 propel the fine mist up vapor tubes 560 into the air foils, directing the vapor into the chamber 550 where the vapor completely saturates the inside of the chamber 550. Any vapor particles remaining in the chamber 550 after the sanitizing is complete are drawn by a suction fan 540 through a vapor return passage back into the reservoir 580 where the vapor undergoes condensation, returning to liquid form. The previously-described problem with commercial hand dryers collecting water which can harbor bacteria is avoided because the vapor that returns to the reservoir 580 is sanitizing solution, not plain water.

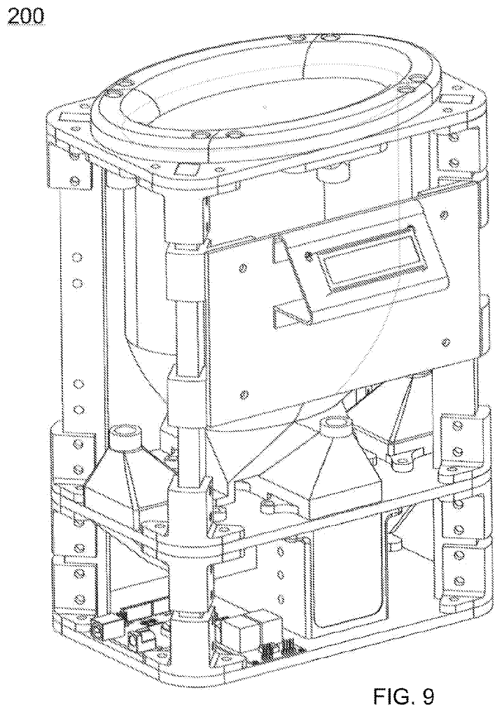

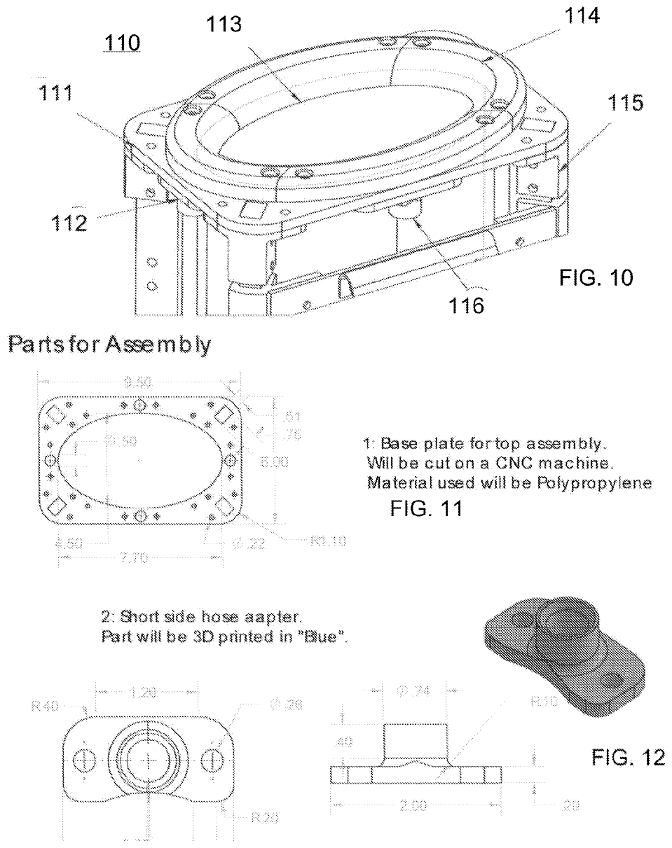

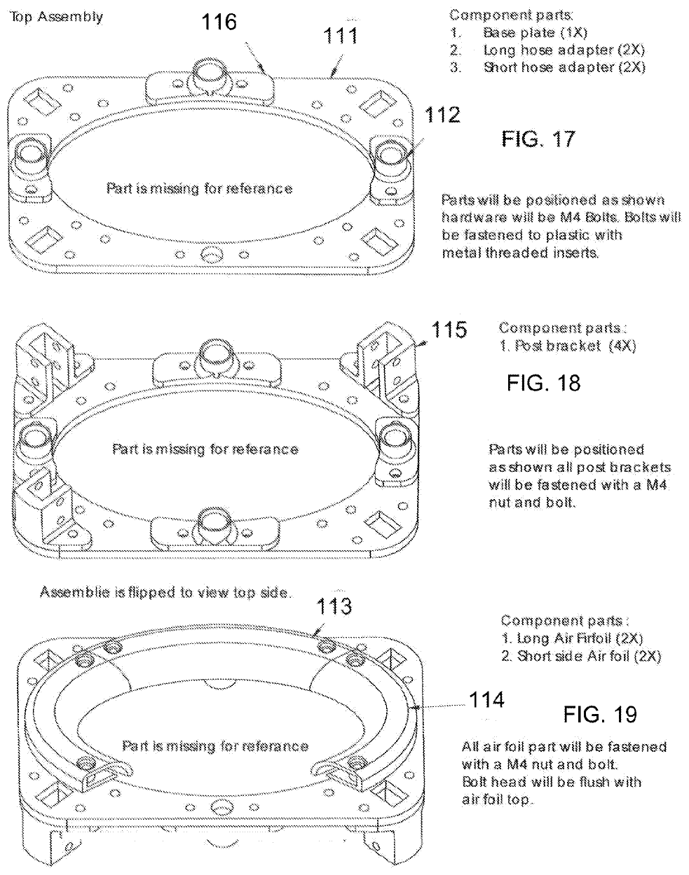

FIG. 9 shows a partial cut-away view of the hand sanitizer 200. FIG. 10 shows the top assembly 110 of the hand sanitizer of FIG. 9, and FIGS. 10-19 show the components of the top assembly 110. The top assembly 110 shown in FIGS. 17-19 is shown flipped upside down in order to demonstrate how the parts are attached. The component parts will be positioned as shown. In one embodiment, bolts will be fastened to the plastic components with metal threaded inserts. The components that are shown are: the base plate 111, the short side hose adapter 112, the short side air foil 114, the long side air foil 113, the long side hose adapter 116, and the post bracket 115.

FIG. 17 is the top assembly 110 showing the base plate 112, two short-side hose adapters 114 and one of two long-side hose adapters 115. FIG. 18 is the top assembly 110 showing the placement of the post bracket 118. FIG. 19 is the top assembly 110, now flipped right-side up, showing the placement of the air foils, both long air foils and short air foils. In this illustration, one of the long air foils is missing for reference.

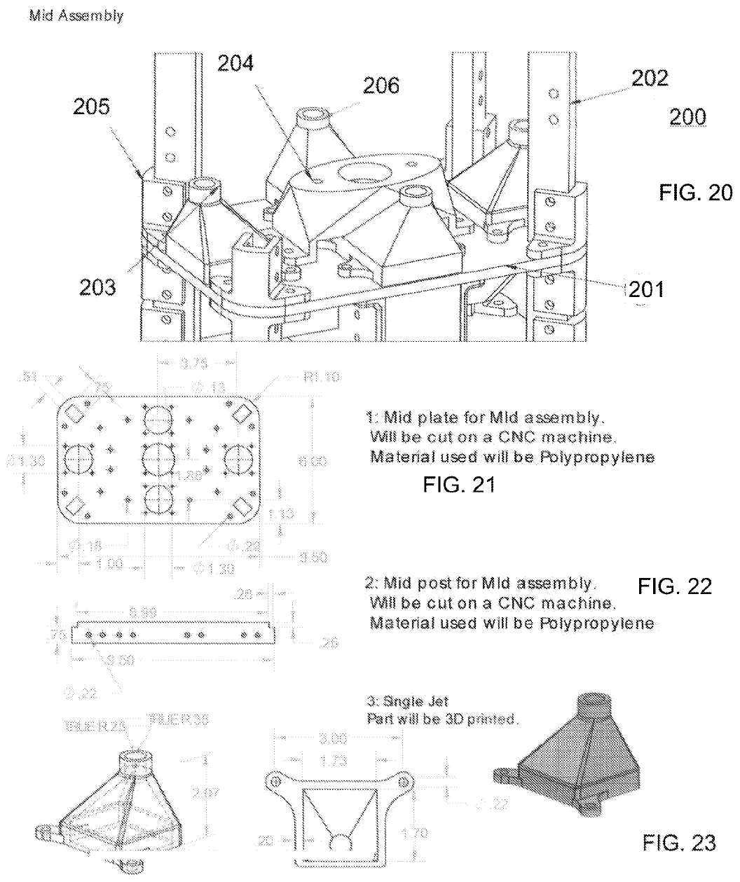

FIG. 20 is a diagram showing the mid-assembly 200 of the hand sanitizer 100. FIG. 21 is a view of the mid plate 201. FIG. 22 shows the post used to attach the mid plate 201. FIGS. 23A, 23B, and 23C show three views of a single jet 203.

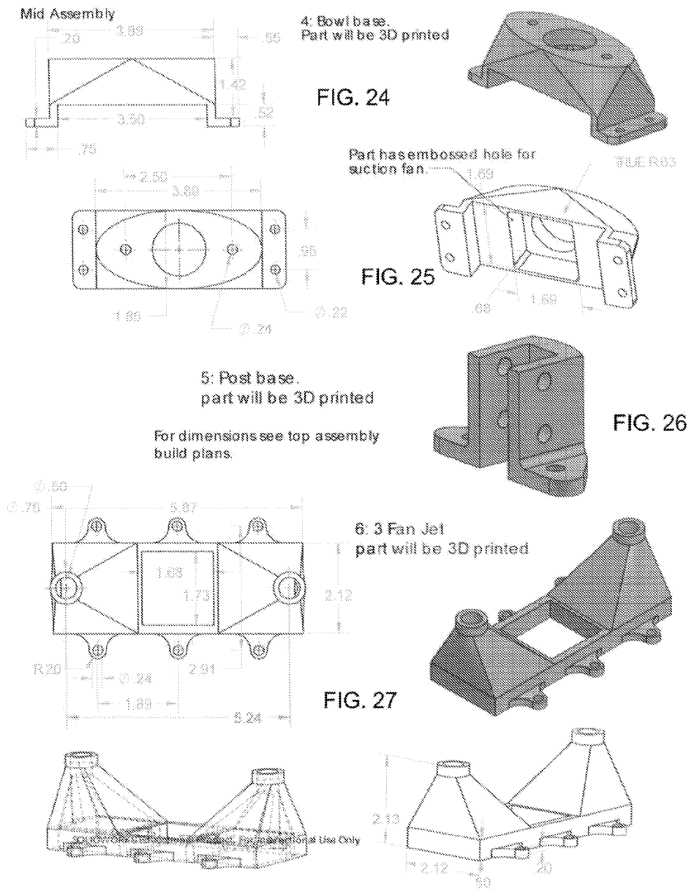

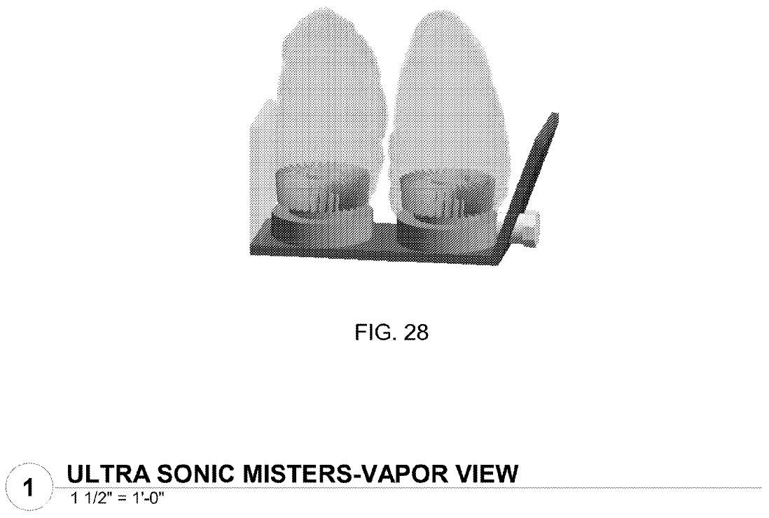

FIG. 24 shows the bowl base 204 with the aperture configured to accept the suction fan. FIG. 25 shows the under-side of the bowl base 205. Clearly visible is the embossed hole for the suction fan. FIG. 26 shows the post base 205. A fan jet 206 is shown in FIG. 27. In the embodiment depicted in FIG. 9, three fan jets 206 are shown.

One with knowledge in the art will appreciate that the orientation of the chamber 550 can be such that a user is able to horizontally insert a hand or hands, rather than the vertical orientation shown in FIG. 7. The operation of the forced air foils and the suction fans operate to constrain the vapor substantially within the chamber, even while maintaining an opening for the insertion of hands.

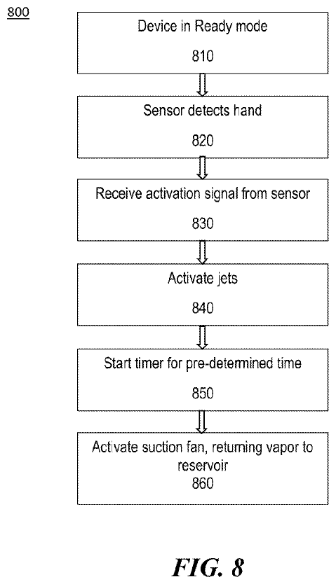

FIG. 8—Operation of hand sanitizer apparatus.

Referring now to FIG. 8 there is shown an operational flow diagram 800 of a method for hand sanitizing according to an embodiment of the present invention. The method begins at step 810 when the electronic circuitry receives a signal that the device has been turned on. In step 820, with the device in Ready mode, the sensor 510 detects that a hand has been placed in the chamber 550. Responsive to sensing the hand(s) in the chamber 550, the sensor 510 sends an activation signal to the electronic circuitry in step 830.

Responsive to receiving the activation signal, in step 840 the electronic circuitry activates the nebulizers, acting on the sanitizing solution in the basin or reservoir 580, generating aerosolized sanitizer in a fine mist. The fan jets propel the vapor to the air foils which direct the vapor to the chamber, saturating the chamber 550 and enveloping the hand(s). The electronic circuitry concurrently starts a timer in step 850 to determine when to de-activate the nebulizers. After a pre-determined period of time, in step 860 the process is reversed. The jets are de-activated and the suction fan 540 is activated, propelling the vapor through the vapor return, back to the reservoir 580. At the end of the sanitizing period, the display screen 515 will display a message to withdraw the hand(s) from the chamber 550. Once the user withdraws his/her hand(s), drying is not required because the sanitizing solution quickly evaporates. In one non-limiting example, the vapor release lasts for approximately 10 seconds after detection of a hand or hands in the chamber 550, at which point the fans are de-activated and the system returns to Ready mode. The excess vapor (the vapor that does not cling to the hand) is pulled by the suction fan 540 into the reservoir 580 where it undergoes condensation, returning to liquid form. The screen 515 can display instructions to the user so that the user knows when the device is ready to sanitize and when to insert and remove hand(s). As an example, the screen 515 can display a countdown to let the user know when sanitization is complete.

### Second Embodiment

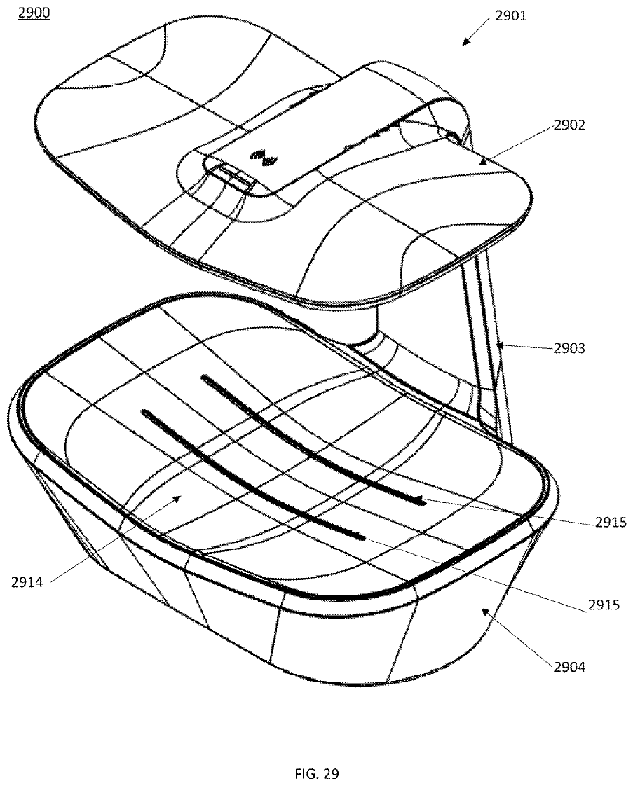

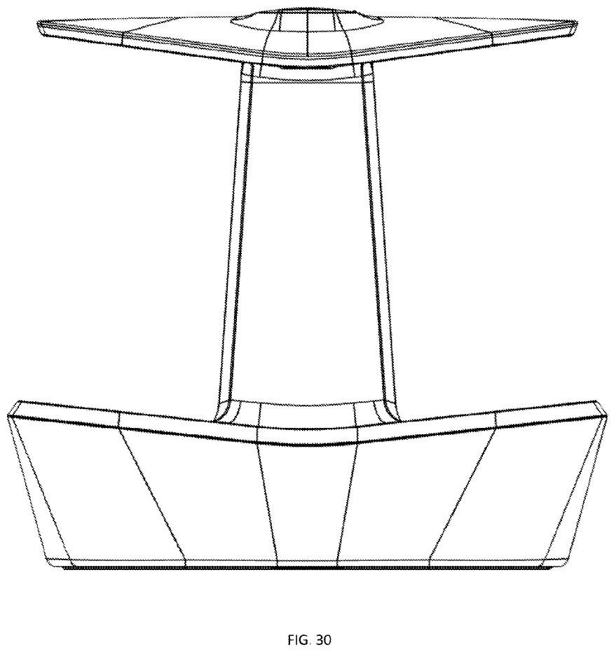

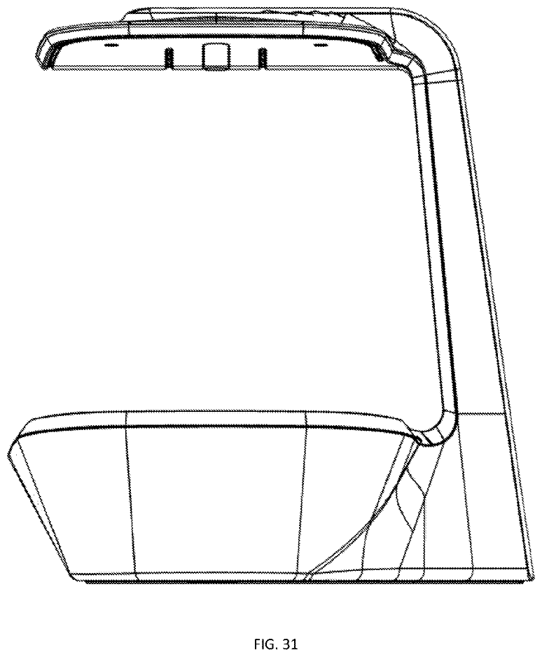

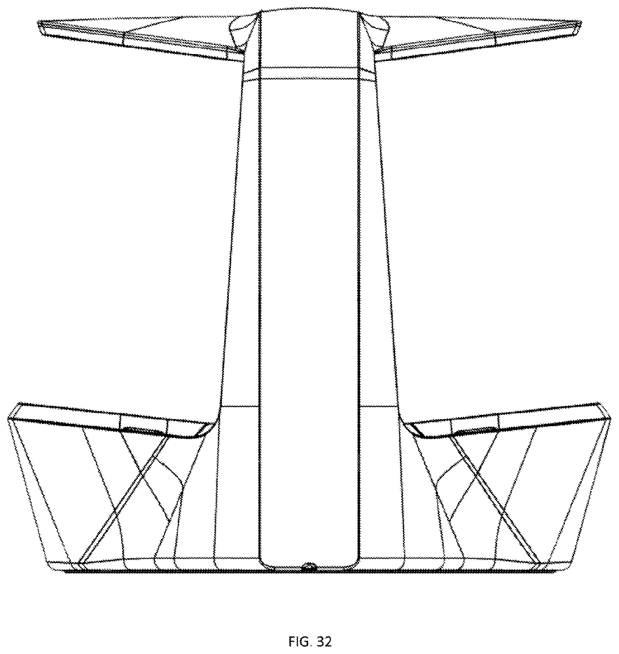

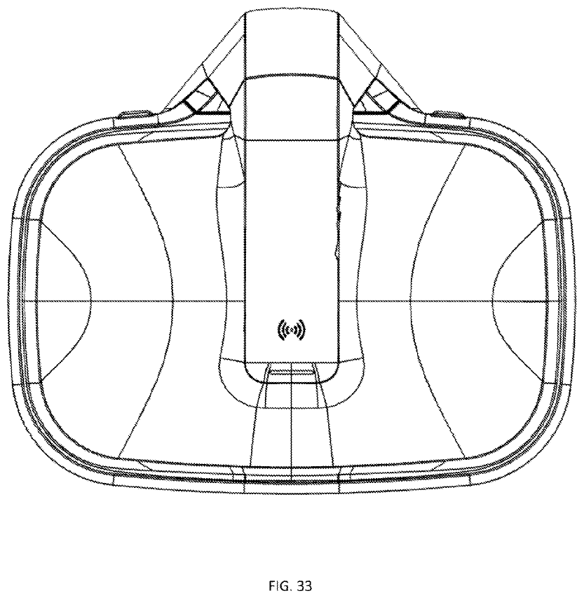

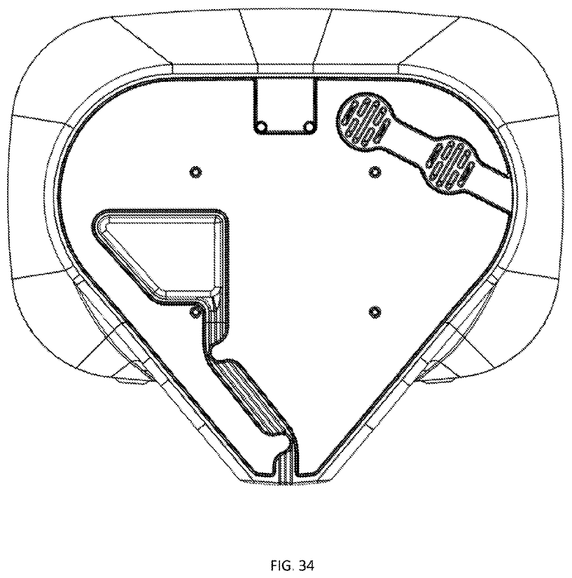

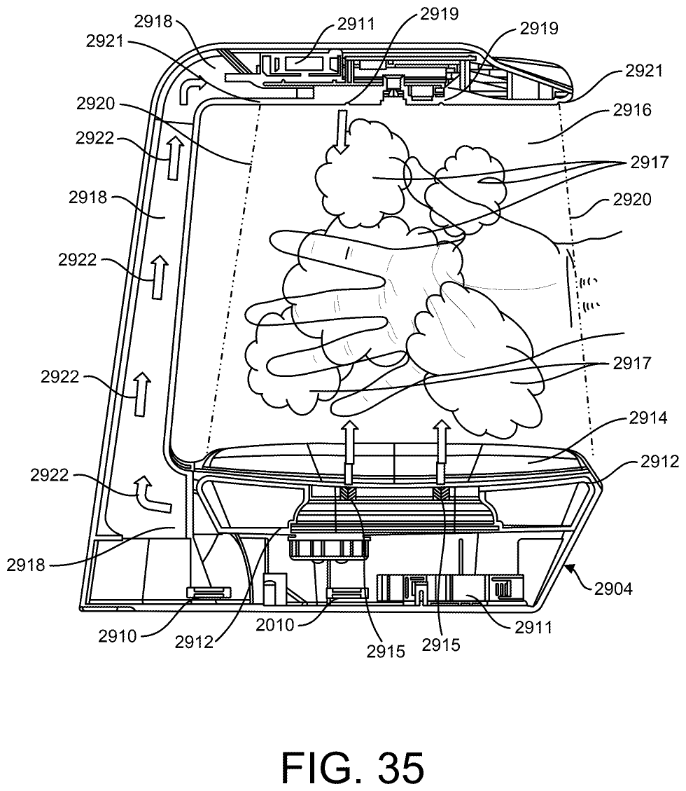

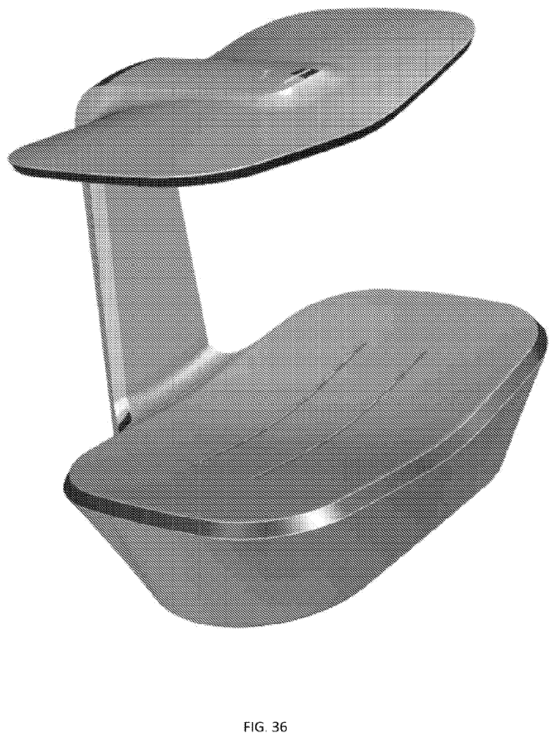

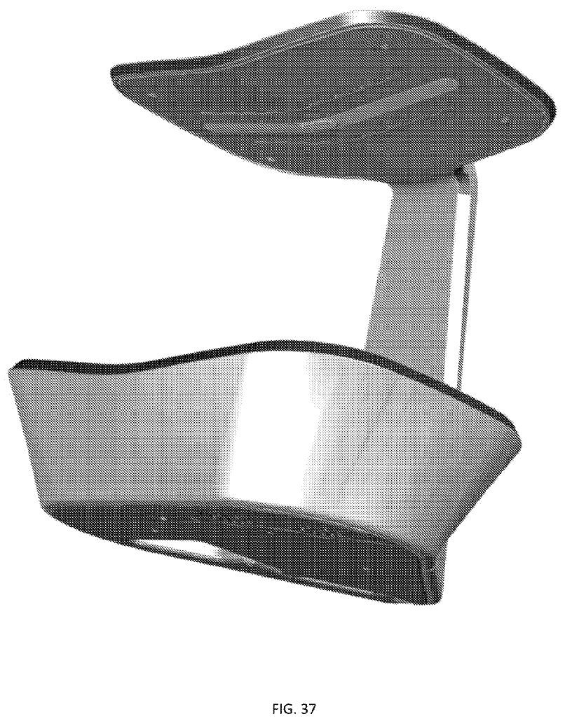

In a second non-limiting embodiment, illustrated in FIGS. 29-39, we describe an embodiment of the apparatus for the delivery of a mist-borne solution substantially contained within a predefined volume where the mist-borne solution is a specially-formulated hand sanitizer and the predefined volume is configured to substantially enclose one or more hands, once inserted therein (see, for example, FIG. 35), primarily utilizing an air curtain for the containment of the mist-borne solution.

Referring now to FIGS. 29-39 in general, and FIG. 29, in particular, we describe apparatus 2900. The apparatus 2900 has a housing 2901 with a head 2902, a neck 2903, and a base 2904. All of the electronics, fans, piezoelectric transducers, as well as, the sanitizing solution, are housed within the housing 2901.

Referring now to FIG. 35, the base 2904 contains the piezoelectric transducers 2910, fans 2911, a replaceable cartridge 2912 containing the sanitizing solution in a liquid state, and an electronics control unit (ECU) 2913 (not shown). The replaceable cartridge 2912 is configured such that, once installed into the base 2904, the sanitizing solution is deposited within the base and in contact with the one or more piezoelectric transduces 2910. The base 2904 is formed with a concave top surface 2914 which includes vent holes 2915 for the mist-borne solution 2917 to pass up into the predefined volume 2916.

The head 2902 is disposed above the base 2904 and is fixedly attached to the base 2904 by the neck 2903. The head 2902, neck 2903, and base 2904 are all in fluid communication with each other through mated openings forming a duct 2918. The upper end of the duct terminates in the head 2092 at another set of vent holes 2919. The head 2902 contains at least one fan 2911. An air curtain 2920 is produced by forcibly directing a high volume of air through perimeter vents 2921 in the head 2902. Also contained in the head 2902 is at least one sensor 2922 configured to detect the presence of a hand within the predefined volume 2916.

In operation, apparatus 2900 is controlled by a specially programmed microcontroller that is part of the ECU 2913 and powered by either batteries or directly connected to an external power supply. The sensor 2922 monitors the predefined volume 2916 for the presence of a hand (or other object as may be required). Once the sensor 2922 detects the presence of a hand (or other object) within the predefined volume 2916, the microcontroller signals the fans 2911 to turn on. The operation of the fans creates an airflow 2922, as well as, the air curtain 2920. The microcontroller also activates the one or more piezoelectric transducer 2910, which in turn, nebulizes the sanitizing solution into a mist-borne solution 2917.

As the mist-borne solution 2917 builds up within the base 2904, it is expelled up through the base vents 2915 into the predefined volume 2916. Additionally, the airflow 2922 draws the mist-borne solution 2917 through the duct 2918, up to the head 2902, where it is expelled into the predefined volume 2916 through the top vents holes 2919. In this way the mist-borne solution 2917 is vented into the predefined volume 2917 both from the top and the bottom, thereby providing full coverage of the inserted hand. The air curtain 2920 substantially keeps the mist-borne solution 2917 from escaping the predefined volume 2916.

At the end of a predetermined time interval, or when the sensor no longer detects a hand within the predefined volume 2916, the microcontroller deactivates the piezoelectric transducers 2910, as well as, the fans 2911. The apparatus 2900 then resumes waiting to detect another incursion into the predefined volume 2916 in order to begin the sanitizing cycle again.

### Third Embodiment

In another embodiment, sanitizing is optimized with the use of ultra violet (UV) lights. Referring to FIG. 7, the hand chamber walls have UV lights 790 that also help kill bacteria during the treatment. The reservoir 580 is also equipped with UV lights that continue killing bacteria. The cartridge 2912 can also be fitted to receive UV lights as well as the interior duct and housing areas.

### Fourth Embodiment

FIGS. 5, 6, and 9 show an embodiment that is integrated with a personnel compliance system, which is activated by using an ID card or key fob. In an embodiment, the hand sanitizer 200 also includes a screen display 515. The screen display 515 can be combined with an NFC (near field communication) reader. In the case of apparatus 2900, an NFC sensor is place in the head 209 of the apparatus. An integrated NFC ID system allows personnel compliance which is activated by using an ID card or a key fob. The user information appears on the systems screen showing their name, date, and time of their hands being sanitized. This information is transferred to an office computer to register the personnel information. Any non-registered person may also use the system, which makes this a unique system for many industries, not only medical.

### Fifth Embodiment

While the invention disclosed herein is capable of delivering into the predefined volume any liquid solution that is capable of being misted, or nebulized, by the piezoelectric transducers, embodiments of the invention are specifically directed to liquid solutions for the sanitizing of hands. The hand sanitizing liquid is preferably a non-alcohol based solution that continues to kill bacteria on hands for up to 4 hours. The sanitizing liquid can be poured directly into the chamber 550; or introduced via a container or bladder coupled with the sanitizer 200 via a proprietary spout that is releasably engaged with the sanitizer 200. The diameter of the spout controls an amount of liquid that flows into the device 200 and the proprietary shape of the spout assures that only the correct solution is introduced into the device 200.

### Sixth Embodiment

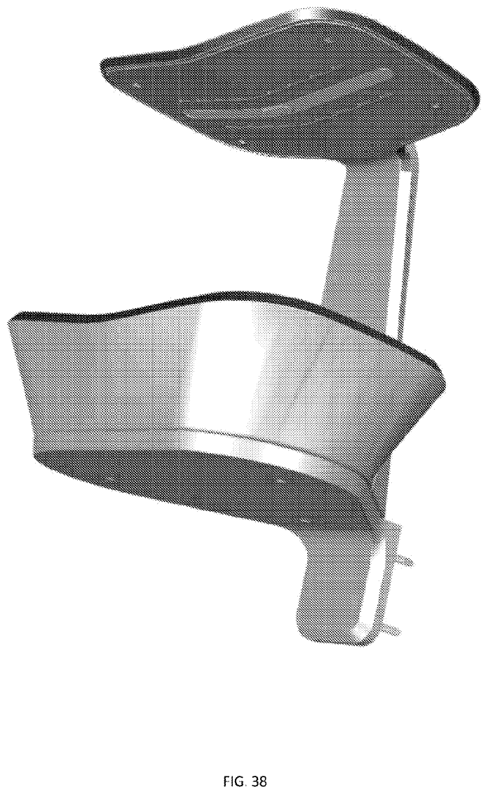

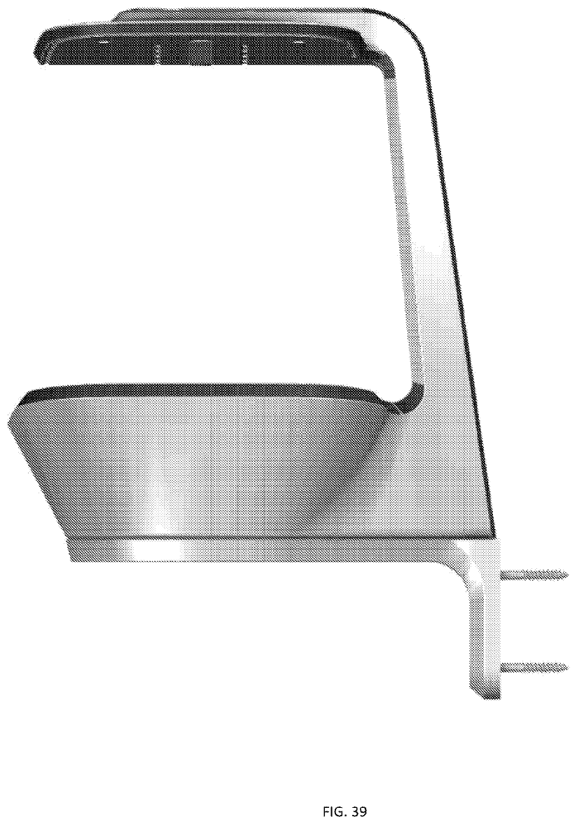

Referring now to FIGS. 38 and 39, we disclose a sixth embodiment of the apparatus that further comprises an integral wall mount bracket.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, although do not preclude the presence or addition of one or more other features, integers, steps, operations, elements, components, and/or groups thereof.

The description of the present application has been presented for purposes of illustration and description, but is not intended to be exhaustive or limited to the invention in the form disclosed. Many modifications and variations will be apparent to those of ordinary skill in the art without departing from the scope and spirit of the invention. The embodiments were chosen and described in order to best explain the principles of the invention and the practical application, and to enable others of ordinary skill in the art to understand various embodiments of the present invention, with various modifications as are suited to the particular use contemplated.

We claim:

1. An apparatus for the delivery of a mist-borne solution substantially contained within a predefined volume, said apparatus comprising:
a predefined volume;
a detection sensor configured to detect the presence of an object within the predefined volume;
a chamber communicative with the predefined volume, configured to contain a solution in a liquid state;
at least one piezoelectric transducer in ultrasonic communication with the solution; and
a micro-controller, in electrical communication with the detection sensor and the at least one piezoelectric transducer, programmed to continuously monitor the predefined volume using the detection sensor to determine the presence of an object within the predefined volume, wherein the micro-controller, upon determining the presence of an object within the predefined volume, activates the at least one piezoelectric transducer thereby vibrating the solution into a mist-borne state such that the mist-borne solution is substantially contained within the predefined volume and surrounding the object within the predefined volume.

2. The apparatus of claim 1 further comprising a housing having a base, a head disposed above the base, and a neck fixedly attached between the head and the base.

3. The apparatus of claim 2 wherein the base, the neck, and the head are in fluid communication via an internally disposed duct.

4. The apparatus of claim 3 wherein the mist-borne solution is at least partially expelled through a one or more base vent, and also at least partially dispersed through the internally disposed duct and expelled through a one or more head vent.

5. The apparatus of claim 4, further comprising at least one fan in communication with a perimeter vent, fan and the perimeter vent configured to produce an air curtain substantially surrounding the predefined volume.

6. The apparatus of any one of the preceding claims further comprising UV lights.

7. The apparatus of any one of the preceding claims further comprising a personnel compliance system.

8. The apparatus of any one of the preceding claims wherein the solution consist essentially of benzalkonium chloride.

9. A method of delivering a mist-borne solution substantially contained within a predefined volume, said method comprising the steps of:
providing an apparatus comprising:
a predefined volume;
a detection sensor configured to detect the presence of an object within the predefined volume;
a chamber communicative with the predefined volume, configured to contain a solution in a liquid state;
at least one piezoelectric transducer in ultrasonic communication with the solution; and
a micro-controller, in electrical communication with the detection sensor and the at least one piezoelectric transducer;
continuously monitoring of the predefined volume by the detection sensor;
detecting the presence of an object within the predefined volume by the detection sensor;
activating by the microcontroller the at least one piezoelectric transducer;
vibrating the solution into a mist-borne state by the at least one piezoelectric transducer; and
saturating the predefined volume with the mist-borne solution, surrounding the object,
whereby, the apparatus delivers the mist-borne solution to the object within the predefined volume.
